(19) United States
(12) Patent Application Publication
(10) Pub. No.: US 2021/0118570 A1
(43) Pub. Date: Apr. 22, 2021

(54) METHODS FOR SCREENING A SUBJECT FOR THE RISK OF CHRONIC KIDNEY DISEASE AND COMPUTER-IMPLEMENTED METHOD

(71) Applicant: Roche Diabetes Care, Inc., Indianapolis, IN (US)

(72) Inventors: Wolfgang Petrich, Mannheim (DE); Tony Huschto, Mannheim (DE); Bernd Schneidinger, Mannheim (DE); Stefan Ravizza, Ehningen (DE); Alexander Buesser, Ehningen (DE)

(73) Assignee: Roche Diabetes Care, Inc., Indianapolis, IN (US)

(21) Appl. No.: 17/040,620

(22) PCT Filed: Mar. 22, 2019
(86) PCT No.: PCT/EP2019/057297

§ 371 Date: Sep. 23, 2020

(30) Foreign Application Priority Data
Mar. 23, 2018 (EP) 18163573.1
Jan. 7, 2019 (EP) 19150615.3

(51) Int. Cl. G16H 50/30; G16B 25/00; G06N 3/08

(52) U.S. Cl. CPC G16H 50/30; G06N 3/08; G16B 25/00

(57) ABSTRACT

The disclosure relates to a method for screening a subject for the risk of chronic kidney disease (CKD), comprising: receiving marker data indicative for a plurality of marker parameters for a subject, such plurality of marker parameters indicating, for the subject for a measurement period, an age value, a sample level of creatinine, and a sample level of albumin; and determining a risk factor indicative of the risk of suffering CKD for the subject from the plurality of marker parameters, wherein the determining comprises: weighting the age value higher than the sample level of albumin, and weighting the sample level of creatinine higher than the sample level of albumin. Further, a computer-implemented method for screening a subject and a method for screening a subject for the risk of chronic kidney disease (CKD) are provided.

The present invention refers to methods for screening a subject for the risk of chronic kidney disease and a computer-implemented method.

### BACKGROUND

In chronic kidney disease (CKD), kidney function is progressively lost, beginning with a decline in the glomerular filtration rate and/or albuminuria and progressing to end-stage renal disease. As a result, dialysis or renal transplant may be necessary (see Unger, J., Schwartz, Z., Diabetes Management in Primary Care, 2nd edition. Lippincott Williams & Wilkens, Philadelphia, USA, 2013). CKD is an serious problem, with an adjusted prevalence of 7% in 2013 (Glassock, R. J. et al., The global burden of chronic kidney disease: estimates, variability and pitfalls, Nat Rev Nephrol 13, 104-114, 2017). The early recognition of CKD could slow progression, prevent complications, and reduce cardiovascular-related outcomes (Platinga, L. C. et al., Awareness of chronic kidney disease among patients and providers, Adv Chronic Kidney Dis 17, 225-236, 2010). CKD may be a microvascular long-term complication of diabetes (Fioretto, P. et al., Residual micro-vascular risk in diabetes: unmet needs and future directions, Nat Rev Endocrinol 6, 19-25, 2010).

Algorithms for risk prediction of CKD by diabetic patients have been published, for example, by Dunkler et al. (Dunkler, D. et al., Risk Prediction for Early CKD in Type 2 Diabetes, Clin J Am Soc Nephrol 10, 1371-1379, 2015), Vergouwe et al. (Vergouwe, Y. et al., Progression to microalbuminuria in type 1 diabetes: development and validation of a prediction rule, Diabetologia 53, 254-262, 2010), Keane et al. (Keane, W. F. et al., Risk Scores for Predicting Outcomes in Patients with Type 2 Diabetes and Nephropathy: The RENAAL Study, Clin J Am Soc Nephrol 1, 761-767, 2006) and Jardine et al (Jardine, M. J. et al., Prediction of Kidney-Related Outcomes in Patients With Type 2 Diabetes, Am J Kidney Dis. 60, 770-778, 2012). Such published algorithms are derived from data originating from major clinical studies.

Further models for risk prediction of CKD have been described for example by Adler Perotte et al. (Adler Perotte et al.:“Risk prediction for chronic kidney disease progression using heterogeneous electronic health record data and time series analysis”, Journal of the American Medical Informatics Association, vol. 22, no. 4, 20 Apr. 2015 (2015 Apr. 20), pages 872-880), Paolo Fraccaro et al. (Paolo Fraccaro et al.: “An external validation of models to predict the onset of chronic kidney disease using population-based electronic health records from Salford, UK”, BMC Medicine, vol. 14, no. 1, 12 Jul. 2016 (2016 Jul. 12), and Justin B. Echouffo-Tcheugui et al. (Justin B. Echouffo-Tcheugui et al.: “Risk Models to Predict Chronic Kidney Disease and Its Progression: A Systematic Review”, Plos Medicine, vol. 9, no. 11, 20 Nov. 2012 (2012 Nov. 20), page e1001344).

Such predictive models based on clinical data represent an ideal setting with a preselected population, cross-checked and validated clinical data entries and often a narrow time window of observation. The outcomes therefore do not necessarily reveal the optimum pathways in terms of efficacy and effectiveness for a real-world population when inferred from clinical studies. In addition, most literature is focused on progression of diabetic nephropathy or CKD and therefore misses the early phase of this diabetic complication. Finally, patients are usually selected on the basis of a full set of respective features.

### SUMMARY

It is an object to provide improved methods for screening a subject for the risk of chronic kidney disease, allowing an early risk assessment for CKD based on real world data (RWD).

To solve this, methods for screening a subject for the risk of chronic kidney disease (CKD) according to the independent claims 1 and 15, respectively, are provided. Further, a computer-implemented method according to the independent claim 14 is provided. Further embodiments are discloses in the dependent claims.

According to an aspect, a method for screening a subject for the risk of chronic kidney disease (CKD) is provided. The method comprises receiving marker data indicative for a plurality of marker parameters for a subject, such plurality of marker parameters indicating, for the subject for a measurement period, an age value, a sample level of creatinine, and a sample level of albumin; and determining a risk factor indicative of the risk of suffering CKD for the subject from the plurality of marker parameters. The determining comprises weighting the age value higher than the sample level of albumin, and weighting the sample level of creatinine higher than the sample level of albumin.

According to another aspect, a computer-implemented method for screening a subject for the risk of chronic kidney disease (CKD) in a data processing system is provided, the data processing system having a processor and a non-transitory memory storing a program causing the processor to execute:
receiving marker data indicative for a plurality of marker parameters for a subject, such plurality of marker parameters indicating, for the subject for a measurement period, an age value, a sample level of albumin, and a sample level of creatinine; and
determining a risk factor indicative of the risk suffering CKD for the subject from the plurality of marker parameters, wherein the determining comprises
weighting the age value higher than the sample level of albumin, and
weighting the sample level of creatinine higher than the sample level of albumin.

According to a further aspect, a method for screening a subject for the risk of chronic kidney disease (CKD) is provided. The method comprises receiving marker data indicative for a plurality of marker parameters, such plurality of marker parameters indicating an age value for the subject, a sample level of creatinine for a measurement period, and a sample level of albumin for a measurement period; and determining a risk factor indicative of the risk of suffering CKD for the subject from the plurality of marker parameters. The determining comprises weighting the age value higher than the sample level of albumin, and weighting the sample level of creatinine higher than the sample level of albumin. At least one of the sample level of creatinine and the sample level of albumin is indicative of a generalized value of sample levels for a reference group of subjects not comprising the subject, for a respective measurement period of each subject of the reference group of subjects.

With regard to such method, for each subject of the reference group of subjects, the measurement period may be limited to two years and may end with a diabetes diagnosis of the respective subject of the reference group of subjects.

For the marker data, screening or determining of outlier values may be performed prior to determining the risk value. In case of determining an outlier (e.g. by checking whether the value exceeds a specific range allowed for that value), the value may be substituted by a value within (expected) standard deviation or by the upper or lower limit of a specific allowable range for that feature. For example, by mistake in the process of collecting the data a value may be provided with a wrong decimal place by the person inputting data. Such value obviously wrong can be corrected. E.g., if the feature value is higher than the upper limit of the specific allowable range for that feature, the value can be replaced by the upper limit of that range before using it in the prediction formula. If the feature value is lower than the lower limit of the specific allowable range for that feature, the value can be replaced by the lower limit before using it in the prediction formula.

For the marker data, screening or determining of missing data or values may be performed prior to determining the risk value. Missing data may be imputed with the cohort's mean value.

One or both of the above measures may be applied for providing improved marker data for determining the risk factor.

A generalized value of sample levels for a reference group of subjects not comprising the subject may be, for example, a maximum value, a minimum value, a mean value, a median value, or a slope determined for a plurality of sample levels for the respective measurement period of each subject of the reference group of subjects. The subjects of the reference group of subjects may be diabetes patients. For example, all subjects of the reference group of subjects may be diabetes patients.

The marker parameters may be indicative of real-world data which is not restricted regarding, for example, completeness or veracity of the data (unlike clinical data).

The age value for the subject for the measurement period may be an age value for the subject at the end of the measurement period.

Within the meaning of the present disclosure, weighting a first value or sample level higher than a second value or sample level means that the first value or sample level and the second value or sample level are used in an equation, such as an equation for determining a risk factor, in such a way that a relative change in the first value or sample level (for example a change of 10% in the first value) influences the result of the equation (for example the risk factor) more than the same relative change in the second value or sample level (in the example above, a change of 10% in the second value). For example, weighting may comprise multiplying the first value or sample level and the second value or sample level with appropriate respective constants. Depending on the expected first value or sample level and the expected second value or sample level and their respective units, weighting the first value or sample level higher than the second value or sample level may comprise multiplying the first value or sample level with a higher or smaller constant than the second value or sample level.

The method may further comprise the plurality of marker parameters indicating, for the subject, a blood sample level of creatinine. Thus, requesting the sample level of creatinine as a concentration in urine may be avoided. The plurality of marker parameters may indicate, for the subject, a selected blood sample level of creatinine selected from a plurality of blood sample levels of creatinine. For example, the selected blood sample level of creatinine may be a maximum value from the plurality of blood sample levels of creatinine. Alternatively or additionally, the plurality of marker parameters may indicate, for the subject, a calculated blood sample level of creatinine calculated from a plurality of blood sample levels of creatinine. For example, the calculated blood sample level of creatinine may be a statistical value calculated from the plurality of blood sample levels of creatinine, such as a mean value.

The sample level of creatinine may be provided in units of mg/dl (such as milligrams of creatinine per deciliter of blood).

The method may further comprise the plurality of marker parameters indicating, for the subject, a blood sample level of albumin. Thus, requesting the sample level of albumin as a concentration in urine may be avoided. The plurality of marker parameters may indicate, for the subject, a selected blood sample level of albumin selected from a plurality of blood sample levels of albumin. For example, the selected blood sample level of albumin may be a minimum value from the plurality of blood sample levels of albumin. Alternatively or additionally, the plurality of marker parameters may indicate, for the subject, a calculated blood sample level of albumin calculated from a plurality of blood sample levels of albumin. For example, the calculated blood sample level of albumin may be a statistical value calculated from the plurality of blood sample levels of albumin, such as a mean value.

The sample level of albumin may be provided in units of g/dl (such as grams of albumin per deciliter of blood).

The subject may be a diabetes patient. Thereby, the risk of chronic kidney disease in a diabetes patient may be screened.

Alternatively, all of the plurality of marker parameters may be for a subject for which a diabetes diagnosis is not available. For example, the subject may be at risk of becoming a diabetes patient. Thereby, the risk of chronic kidney disease in a subject not having been diagnosed with diabetes, for example a subject at risk of becoming a diabetes patient, may be screened. The receiving may comprise receiving marker data indicative for a plurality of marker parameters for the subject for which a diabetes diagnosis is not available.

The measurement period may be limited to two years. Thereby, values and/or sample levels of substances may be provided that have been collected within a time period of a maximum of two years with the risk factor indicating a risk of suffering CKD for the subject from the end of the measurement period onwards.

The subject may not have been diagnosed with diabetes by the end of the measurement period. For example, the risk of CKD may be screened in a subject that has recently been diagnosed with diabetes and the marker data may be indicative for a plurality of marker parameters for the subject for a measurement period that lies entirely before the diabetes diagnosis for the subject. Alternatively, the risk of CKD may be screened for a subject that has not been diagnosed with diabetes at all, the marker data therefore being indicative for a plurality of marker parameters for the subject for a measurement period in which the subject has not been diagnosed with diabetes.

The measurement period may lie after a diabetes diagnosis for the subject, at least in part. For example, at most 20% of the measurement period, preferably at most 10% of the measurement period, may lie after a time at which the subject was diagnosed with diabetes. For example, the subject may be a diabetes patient who has been diagnosed with diabetes for less than two years and the marker data may be indicative for a plurality of marker parameters for the patient for a measurement period, such as a measurement period of two years, that ends directly or shortly prior to the determining the risk factor, such that part of the plurality of marker parameters is for a time period before the diabetes diagnosis for the patient and part of the plurality of marker parameters is for a time period after the diabetes diagnosis for the patient.

The measurement period may lie entirely after a diabetes diagnosis for the diabetes patient. For example, the subject may be a diabetes patient who has been diagnosed with diabetes for more than two years and the marker data may be indicative for a plurality of marker parameters for the patient for a measurement period, such as a measurement period of two years, that ends directly or shortly prior to the determining the risk factor.

The risk factor may be indicative of the risk of suffering CKD for the subject within a prediction time period of three years from the end of the measurement period. The risk factor may be a probability for the subject of developing CKD within three years from the time the last value and/or sample level has been determined. Alternatively, the risk factor may be indicative of the risk of suffering CKD for the subject within a time period of less than three years, for example two years, from the end of the measurement period. As a further alternative, the risk factor may be indicative of the risk of suffering CKD for the subject within a time period of more than three years from the end of the measurement period.

The determining may further comprise weighting the age higher than the sample level of creatinine.

According to the aforementioned, the marker parameters include an age value, a sample lev-el of creatinine and a sample level of albumin, thereby providing a simple method for calculating a risk factor indicative of the risk of suffering CKD. In further embodiments, as will be set forth in more detail below, further marker parameters including at least one of a sample level of estimated glomerular filtration rate, a body mass index, a sample level of glucose and a sample level of HbA1c may optionally be included in the risk calculation.

The receiving may comprise receiving marker data indicative for a plurality of marker parameters for a subject having a sample level of HbA1c of less than 6.5%. HbA1C is the C-fraction of glycated haemoglobin A1. The sample level of HbA1c may be provided in units of % (such as a percentage in blood). Alternatively, the sample level of HbA1c may be provided in units of mmol/mol (such as mmol of HbA1c per mol of blood).

The method may further comprise the plurality of marker parameters indicating, for the subject, a sample level of a glomerular filtration rate, and in the determining, weighting each of the age value, the sample level of albumin, and the sample level of creatinine higher than the sample level of a glomerular filtration rate.

The plurality of marker parameters may indicate, for the subject, a selected glomerular filtration rate selected from a plurality of glomerular filtration rates. For example, the selected glomerular filtration rate may be a minimum value from the plural glomerular filtration rates. Alternatively or additionally, the plurality of marker parameters may indicate, for the subject, a calculated glomerular filtration rate calculated from a plurality of glomerular filtration rates. For example, the calculated glomerular filtration rate may be a statistical value calculated from the plurality of glomerular filtration rates, such as a mean value.

The glomerular filtration rate is known in the art to be indicative of the flow rate of filtered fluid through the kidney and is an important indicator for estimating renal function. The glomerular filtration rate may decrease due to renal disease. In embodiments, the glomerular filtration rate may be estimated using a Modification of Diet in Renal Disease (MDRD) formula, known in the art as such. For example, a MDRD formula using four variables relies on age, sex, ethnicity and serum creatinine of the subject for estimating glomerular filtration rate. In alternative embodiments, the glomerular filtration rate may be estimated using the CKD-EPI (Chronic Kidney Disease Epidemiology Collaboration) formula, known in the art as such. The CKD-EPI formula relies on age, sex, ethnicity and serum creatinine of the subject for estimating glomerular filtration rate. In further embodiments, the glomerular filtration rate may be estimated using other methods or may be directly determined. The sample glomerular filtration rate may be provided in units of ml/min/1.73m² (milliliters per minute per 1.73 square meters of body surface area).

The risk factor (P′_{CKD}) may be determined according to the following equation:

$${P}_{CKD}^{‵} = \frac{{e}^{{P}_{CKD\_Pred}^{‵}}}{1 + {e}^{{P}_{CKD\_Pred}^{‵}}}$$

Herein, P′_{CKD_Pred} may be calculated as

P′_{CKD_Pred}=c′_{CKD1}·age+c′_{CKD2}·creatinine+c′_{CKD3}·albumin+c′_{CKD4,}

wherein age is the age of the subject in years, creatinine is a sample level of creatinine for the subject, albumin is a sample level of albumin for the subject, and c′_{CKD1}, c′_{CKD2}, c′_{CKD3}, and c′_{CKD4} are constants.

In an alternative, the risk factor (P_{CKD}) may be determined according to the following equation:

$${P}_{CKD} = \frac{{e}^{{P}_{CKD\_Pred}}}{1 + {e}^{{P}_{CKD\_Pred}} + {e}^{{P}_{Death\_Pred}}}$$

Herein, P_{CKD_Pred} may be calculated as

P_{CKD_Pred}=c_{CKD1}·age+c_{CKD2}·creatinine+c_{CKD3}·albumin+c_{CKD4}

and P_{Death_Pred} may be calculated as

P_{Death_Pred}=c_{Death1}·age+c_{Death2}·creatinine+c_{Death3}·albumin+c_{Death4},

wherein age is the age of the subject in years, creatinine is a sample level of creatinine for the subject, albumin is a sample level of albumin for the subject, and c_{CKD1}, c_{CKD2}, c_{CKD3}, c_{CKD4}, c_{Death1}, c_{Death2}, c_{Death3} and c_{Death4} are constants. Such formula may be applied in case there is death prediction revealed from the RWD analysis. Otherwise, constants with respect to death prediction may be omitted as outlined above.

The sample level of creatinine may be a sample level of creatinine from a plurality of sample levels of creatinine. The sample level of albumin may be a sample level of albumin from a plurality of sample levels of albumin. The sample level of creatinine and/or the sample level of albumin may be a representative sample level from the respective plurality of sample levels of creatinine and/or albumin, such as a maximum sample level, a minimum sample level, a mean sample level and/or a median of the sample levels. In an exemplary embodiment, creatinine is a maximum sample level of creatinine from a plurality of sample levels of creatinine for the subject and albumin is a minimum sample level of albumin from a plurality of sample levels of albumin for the subject.

The constants c′_{CKD1}, c′_{CKD2}, c′_{CKD3}, and c′_{CKD4} may be model specific constants. In embodiments, the constants c′_{CKD1}, c′_{CKD2}, and c′_{CKD3} may be constant weighting factors associated with the respective marker parameter.

The constants c_{CKD1}, c_{CKD2}, c_{CKD3}, and c_{CKD4}, and c_{Death1}, c_{Death2}, c_{Death3}, and CDeath4 may be model specific constants. In embodiments, the constants c_{CKD1}, c_{CKD2}, and c_{CKD3}, and c_{Death1}, c_{Death2} and c_{Death3} may be constant weighting factors associated with the respective marker parameter.

For example, the constants may be the following:

c_{CKD1}: 0.02739/year;

c_{CKD2}: 1.387 dl/mg;

c_{CKD3}: −0.3356 dl/g; and

c_{CKD4}: −3.1925.

c_{Death1}: 0.06103/year;

c_{Death2}: 0.8194 dl/mg;

c_{Death3}: −0.9336 dl/g; and

c_{Death4}: −3.3325.

In embodiments, any or each of the constants may be selected from a range of +/−30% around such respective value, preferably from a range of +/−20%, and more preferably from a range of +/−10%

The risk factor (P″_{CKD}) may be determined according to the following equation:

$${P}_{CKD}^{‵ ′} = \frac{{e}^{{{P}^{‵ ′}}_{CKD\_Pred}}}{1 + {e}^{{{P}^{‵ ′}}_{CKD\_Pred}}}$$

Herein, P″_{CKD_Pred} may be calculated as

P″_{CKD_Pred}=c_{CKD1}·age+c″_{CKD2}·creatinine+c″_{CKD3}·albumin+c″_{CKD4}+c″_{CKD5}·eGFR.

wherein age is the age of the subject in years, creatinine is a sample level of creatinine for the subject, albumin is a sample level of albumin for the subject, eGFR is a sample level of estimated glomerular filtration rate for the subject, and c″_{CKD1}, c″_{CKD2}, c″_{CKD3}, c″_{CKD4}, and c″_{CKD5}-are constants.

In another example, the risk factor (P′_{CKD}) may be determined according to the following equation:

$${P}_{CKD}^{′} = \frac{{e}^{{P}_{CKD\_Pred}^{′}}}{1 + {e}^{{P}_{CKD\_Pred}^{′}} + {e}^{{P}_{Death\_Pred}^{′}}}$$

Herein, P′_{CKD_Pred} may be calculated as

P′_{CKD_Pred}=c′_{CKD1}·age+c′_{CKD2}·creatinine+c′_{CKD3}·albumin+c′_{CKD4}+c′_{CKD5}·GFR,

and P′_{Death_Pred} may be calculated as

P′_{Death_Pred}=c′_{Death1}·age+c′_{Death2}·creatinine+c′_{Death3}·albumin+c′_{Death4}+c′_{Death5}·eGFR,

wherein age is the age of the subject in years, creatinine is a sample level of creatinine for the subject, albumin is a sample level of albumin for the subject, eGFR is a sample level of estimated glomerular filtration rate for the subject, and c′_{CKD1}, c′_{CKD2}, c′_{CKD3}, c′_{CKD4}, c′_{CKD5}, c′_{Death1}, c′_{Death2}, c′_{Death3}, c′_{Death4} and c′_{Death5} are constants. Such formula may be applied in case there is death prediction revealed from the RWD analysis. Otherwise, constants with respect to death prediction may be omitted as outlined above.

The sample level of creatinine may be a sample level of creatinine from a plurality of sample levels of creatinine. The sample level of albumin may be a sample level of albumin from a plurality of sample levels of albumin.

With regard to the estimated glomerular filtration rate, it may be estimated glomerular filtration rate from a plurality of levels available for the subject.

The sample level of creatinine, the sample level of albumin and/or the sample level of estimated glomerular filtration rate may be a representative sample level from the respective plurality of sample levels of creatinine, albumin and/or estimated glomerular filtration rate, such as a maximum sample level, a minimum sample level, a mean sample level and/or a median of the sample levels. In an exemplary embodiment, creatinine is a maximum sample level of creatinine from a plurality of sample levels of creatinine for the subject, albumin is minimum a sample level of albumin from a plurality of sample levels of albumin for the subject and eGFR is a minimum sample level of estimated glomerular filtration rate from a plurality of sample levels of estimated glomerular filtration rate for the subject.

The constants c″_{CKD1}, c″_{CKD2}, c″_{CKD3}, c″_{CKD4} and c″_{CKD5} may be model specific constants. In embodiments, the constants c″_{CKD1}, c″_{CKD2}, and c″_{CKD3} and c″_{CKD5} may be constant weighting factors associated with the respective marker parameter.

The constants c′_{CKD1}, c′_{CKD2}, c′_{CKD3}, c′_{CKD4} and c′_{CKD5}, and c_{Death1}, c′_{Death2}, c′_{Death3}, c′_{Death4} and c′_{Death5} may be model specific constants. In embodiments, the constants c′_{CKD1}, c′_{CKD2}, c′_{CKD3}, and c′_{CKD5}, and c′_{Death1}, c′_{Death2}, c′_{Death3}, and c′_{Death5} may be constant weighting factors associated with the respective marker parameter.

In such embodiment, for example, the constants may be the following:

c′_{CKD1}: 0.02739/year;

c′_{CKD2}: 1.387 dl/mg;

c′_{CKD3}: −0.3356 dl/g;

c′_{CKD4}: −1.3013; and

c′_{CKD5}: −0.02843 min·1.73 m²/ml.

c′_{Death1}: 0.06103/year;

c′_{Death2}: 0.8194 dl/mg;

c′_{Death3}: −0.9336 dl/g;

c′_{Death4}: −4.4328; and

c′_{Death5}: 0.01654 min·1.73 m²/ml.

In embodiments, any or each of the constants may be selected from a range of +/−30% around such respective value, preferably from a range of +/−20%, and more preferably from a range of +/−10%

In further embodiments, the risk factor (P′″_{CKD}) may be determined according to the following equation:

$${P}_{CKD}^{‵ ″} = \frac{{e}^{{P}_{CKD\_Pred}^{‵ ″}}}{1 + {e}^{{P}_{CKD\_Pred}^{‵ ″}}}$$

Herein, P′″_{CKD_Pred} may be calculated as

P″_{CKD_Pred}=c′″_{CKD1}·age+c′″_{CKD2}·creatinine+c′″_{CKD3}·albumin+c′″_{CKD4}+c′″_{CKD5}·eGFR+c′″_{CKD6}·BMI+c′″_{CKD7}·Glucose+c′″_{CKD8}·HbA1c.

wherein age is the age of the subject in years, creatinine is a sample level of creatinine for the subject, albumin is a sample level of albumin for the subject, eGFR is a sample level of estimated glomerular filtration rate for the subject, BMI is a value of the Body Mass Index (BMI) for the subject, Glucose is a sample level of glucose for the subject, HbA1c is a sample level of C-fraction of glycated haemoglobin A1 for the subject and c′″_{CKD1}, c′″_{CKD2}, c′″_{CKD3}, c′″_{CKD4}, c′″_{CKD5}, c′″_{CKD6}, c′″_{CKD7}, and c′″_{CKD8} are constants. The BMI may be provided in units of kg/m² (kilograms per square meter) and determined as known in the art. The minimum sample level of glucose may be provided in units of mg/dl (such as milligrams of glucose per deciliter of blood).

In another example, the risk factor (P″_{CKD}) may be determined according to the following equation:

$${P}_{CKD}^{″} = \frac{{e}^{{P}_{C K {D}_{-} P r e d}^{″}}}{1 + {e}^{{P}_{CKD\_Pred}^{″}} + {e}^{{P}_{Death\_Pred}^{″}}}$$

Herein, P″_{CKD_Pred} may be calculated as

P″_{CKD_Pred}=c″_{CKD1}·age+c″_{CKD2}·creatinine+c″_{CKD3}·albumin+c′_{CKD4}+c_{CKD5}·eGFR+c″_{CKD6}·BMI+c″_{CKD7}·Glucose+c″_{CKD8}·HbA1c,

and P″_{Death_Pred} may be calculated as

P″_{Death_Pred}=c″_{Death1}·age+c″_{Death2}·creatinine+c″_{Death3}·albumin+c″_{Death4}+c″_{Death5}·eGFR+c″_{Death6}·BMI+c″_{Death7}·Glucose+c″_{Death8}·HbA1c,

wherein age is the age of the subject in years, creatinine is a sample level of creatinine for the subject, albumin is a sample level of albumin for the subject, eGFR is a sample level of estimated glomerular filtration rate for the subject, BMI is a value of the Body Mass Index (BMI) for the subject, Glucose is a sample level of glucose for the subject, HbA1c is a sample level of C-fraction of glycated haemoglobin A1 for the subject and c″_{CKD1}, c″_{CKD2}, c″_{CKD3}, c″_{CKD4}, c″_{CKD5}, c″_{CKD6}, c″_{CKD7}, c″_{CKD8}, c″_{Death1}, c″_{Death2}, c″_{Death3}, c″_{Death4}, c″_{Death5}, c″_{Death6}, c″_{Death7} and c″_{Death8} are constants. The BMI may be provided in units of kg/m² (kilograms per square meter) and determined as known in the art. The minimum sample level of glucose may be provided in units of mg/dl (such as milli-grams of glucose per deciliter of blood). Such formula may be applied in case there is death prediction revealed from the RWD analysis. Otherwise, constants with respect to death prediction may be omitted as outlined above.

In embodiments, any or each of the constants may be selected from a range of +/−30% around such respective value, preferably from a range of +/−20%, and more preferably from a range of +/−10%

The sample level of creatinine may be a sample level of creatinine from a plurality of sample levels of creatinine for the subject, the sample level of albumin may be a sample level of albumin from a plurality of sample levels of albumin for the subject, the sample level of estimated glomerular filtration rate may be a sample level of estimated glomerular filtration rate from a plurality of sample levels of estimated glomerular filtration rate for the subject, the value of the Body Mass Index (BMI) may be a value of the BMI from a plurality of values of the BMI for the subject, the sample level of glucose may be a sample level of glucose from a plurality of sample levels of glucose for the subject, and/or the sample level of C-fraction of glycated haemoglobin A1 may be a sample level of C-fraction of glycated haemoglobin A1 from a plurality of sample levels of C-fraction of glycated haemoglobin A1 for the subject

The sample level of creatinine, the sample level of albumin, the sample level of estimated glomerular filtration rate, the value of the Body Mass Index, the sample level of glucose, and/or the sample level of C-fraction of glycated haemoglobin Al may be a representative sample level from the respective plurality of sample levels of creatinine, albumin, estimated glomerular filtration rate, Body Mass Index, glucose, and/or C-fraction of glycated haemoglobin A1, such as a maximum sample level, a minimum sample level, a mean sample level and/or a median of the sample levels. In an exemplary embodiment, creatinine is a maximum sample level of creatinine from a plurality of sample levels of creatinine for the subject, albumin is minimum a sample level of albumin from a plurality of sample levels of albumin for the subject, eGFR is a minimum sample level of estimated glomerular filtration rate from a plurality of sample levels of estimated glomerular filtration rate for the subject, .BMI is a minimum value of the Body Mass Index (BMI) from a plurality of values of the BMI for the subject, Glucose is a minimum sample level of glucose from a plurality of sample levels of glucose for the subject, and HbA is a mean sample level of C-fraction of glycated haemoglobin A1 from a plurality of sample levels of C-fraction of glycated haemoglobin A1 for the subject.

The constants c′″_{CKD1}, c′″_{CKD2}, c′″_{CKD3}, c′″_{CKD4}, c′″_{CKD5}, c′″_{CKD6}, c′″_{CKD7}, and c′″_{CKD8}, may be model specific constants. In embodiments, the constants c′″_{CKD1}, c′″_{CKD2}, c′″_{CKD3}, c′″_{CKD5}, c′″_{CKD6}, c′″_{CKD7}, and c′″_{CKD8} may be constant weighting factors associated with the respective marker parameter.

The constants c′″_{CKD1}, c′″_{CKD2}, c′″_{CKD3}, c′″_{CKD4}, c′″_{CKD5}, c′″_{CKD6}, c′″_{CKD7}, and c′″_{CKD8}, and c′″_{Death1}, c′″_{Death2}, c″_{Death3}, c″_{Death4}, c″_{Death5}, c″_{Death6}, c″_{Death7} and c″_{Death8} may be model specific constants. In embodiments, the constants c″_{CKD1}, c″_{CKD2}, c″_{CKD3}, c″_{CKD5}, c″_{CKD6}, c″_{CKD7}, and c″_{CKD8}, and c″_{Death1}, c″_{Death2}, c″_{Death3}, c″_{Death4}, c″_{Death5}, c″_{Death6}, c″_{Death7} and c″_{Death8} may be constant weighting factors associated with the respective marker parameter.

In such embodiment, for example, the constants may be the following:

c″_{CKD1}: 0.02739/year;

c″_{CKD2}: 1.387 dl/mg;

c″_{CKD3}: −0.3356 dl/g;

c″_{CKD4}: −2.409;

c″_{CKD5}: −0.02843 min·1.73 m²/ml;

c″_{CKD6}: 0.01128 m²/kg;

c″_{CKD7}: 0.0004946 dl/mg; and

c″_{CKD8}: 0.0893/%.

c″_{Death1}: 0.06103/year;

c″_{Death2}: 0.8194 dl/mg;

c″_{Death3}: −0.9336 dl/g;

c″_{Death4}: −4.557;

c″_{Death5}: 0.01654 min·1.73 m²/ml;

c″_{Death6}: −0.0101 m²/kg;

c″_{Death7}: 0.0009107 dl/mg; and

c″_{Death8}: 0.04368/%.

In embodiments, any or each of the constants may be selected from a range of +/−30% around such respective value, preferably from a range of +/−20%, and more preferably from a range of +/−10%

In embodiments, for any or all of creatinine, albumin, eGFR, BMI, Glucose and HbA, generalized values (creatinine_{gen}, albumin_{gen}, eGFR_{gen}, BMI_{gen}, Glucose_{gen}, HbA_{gen}) may be used instead of values for the subject. For example, mean values for the general population or mean values for a relevant sub-population may be used. As generalized values, mean values of representative values from a respective plurality of values for each population members may be used, for example mean values of a respective maximum value, a respective minimum value, a respective mean value and/or a respective median of values.

In such embodiments, for example, the generalized values may be the following:

creatinine_{gen}: 1.055 mg/dl;

albumin_{gen}: 3.835 g/dl;

eGFR_{gen}: 66.523 ml/min/1.73m²;

BMI_{gen}: 32.295 kg/m²;

Glucose_{gen}: 129.691 mg/dl; and

HbA_{gen}: 7.607%.

In embodiments, any or each of the generalized values may be selected from a range of +/−30% around such respective value, preferably from a range of +/−20%, and more preferably from a range of +/−10%

The method may further comprise determining a subject value recommendation and providing a recommendation output indicative of the subject value recommendation. The determining the subject value recommendation may comprise determining, based on the weighting of the marker parameters, a first marker parameter for which a generalized value was received and which is weighted higher than a second marker parameter for which a generalized value was received, and determining the subject value recommendation to be a recommendation to acquire a value for the first marker parameter for the subject. The recommendation output may be indicative of an instruction to acquire a value for the first marker parameter for the subject and re-perform the method for screening a subject for the risk of CKD, providing marker data comprising the value for the first marker parameter for the subject.

The method may comprise only determining the subject value recommendation and providing the recommendation output indicative of the subject value recommendation if it is determined that a value of accuracy of the risk factor is below an accuracy threshold. The value of accuracy of the risk factor may be determined based on for which marker parameters, generalized values are used. In embodiments, the value of accuracy of the risk factor may be determined in comparison to a reference risk factor that is determined using values for the subject for all or any of the marker parameters for which generalized values are used when determining the risk factor.

Within the meaning of the present disclosure, screening a subject for the risk of CKD means identifying a subject at risk of developing or having CKD.

A sample level in the sense of the present disclosure is a level of a substance, such as creatinine or albumin, in a sample of a bodily fluid of the subject. Sample levels may be determined in the same or different samples. Alternatively or additionally, for determining sample levels, measurements may be performed in the same or different samples. For example, a sample level of a substance may be determined from a plurality of measurements of the same substance in the same sample, for example by determining a mean value. In another example, at least one of a plurality of sample levels of the same substance may be determined in a first sample and at least another one of the plurality of sample levels of the same substance may be determined in a second sample. A sample level of a first substance and a sample level of a second substance may be determined in the same sample. Alternatively, a sample level of a first substance may be determined in a first sample and a sample level of a second substance may be determined in a second sample.

A computer program product may be provided, including a computer readable medium embodying program code executable by a process of a computing device or system, the program code, when executed, causing the computing device or system to perform the computer-implemented method for screening a subject for the risk of chronic kidney disease.

With regard to the computer-implemented method, the computer program product and the further method for screening a subject for the risk of chronic kidney disease, the alternative embodiments described above may apply mutatis mutandis.

In the computer-implemented method, the sample level of albumin may be a sample level of albumin in a bodily fluid sample and the sample level of creatinine may be a sample level of creatinine in another bodily fluid.

In the computer-implemented method, the program may further cause the processor to execute generating output data indicative of the risk factor and outputting the output data to an output device of the data processing system. The output device may be any device suitable for outputting the output data, for example a display device of the data processing system, such as a monitor, and/or a transmitter device for transmitting for wired and/or wireless data transmission. The output data may be output to a user, for example a physician. The output data may be output via a display of the data processing system.

The data processing system may comprise a plurality of data processing devices, each data processing device having a processor and a memory. The marker data may be provided in a first data processing device. For example, the marker data may be received in the first data processing device by user input via an input device and/or by data transfer. The marker data may be sent from the first data processing device to a second data processing device which may be located remotely with respect to the first data processing device. The marker data may be received in the second data processing device and the risk factor may then be determined in the second data processing device. Result data indicative of the risk factor may be sent from the second data processing device to the first data processing device or, alternatively or additionally, to a third data processing device. The result data may then be stored in the first and/or the third data processing device and/or output via an output device of the first and/or the third data processing device.

The first data processing device and/or the third data processing device may be a local device, such as a client computer, and the second data processing device may be a remote device, such as a remote server.

Alternatively, the functionality of at least the first data processing device and the second data processing device may be provided in the same data processing device, for example a computer, such as a computer in a physician's office. All steps of the computer-implemented method may be executed in the same data-processing device.

### DESCRIPTION OF FURTHER EMBODIMENTS

Following, further embodiments are described by way of example. In the figures show:

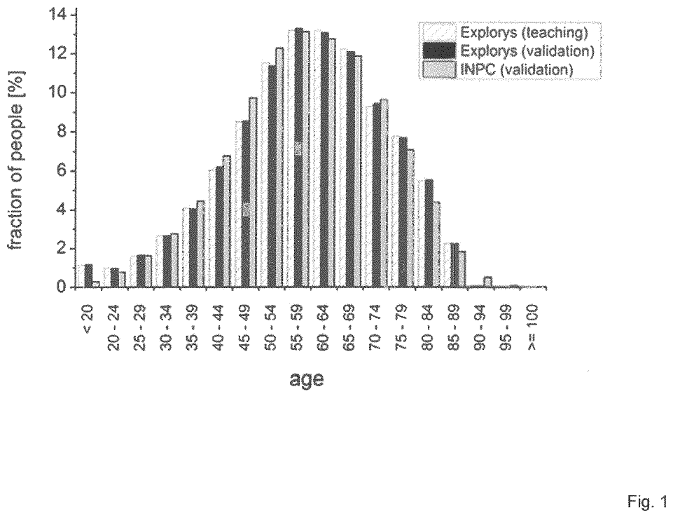

FIG. 1 the distribution of age in an example teaching training set, validation set and further validation set;

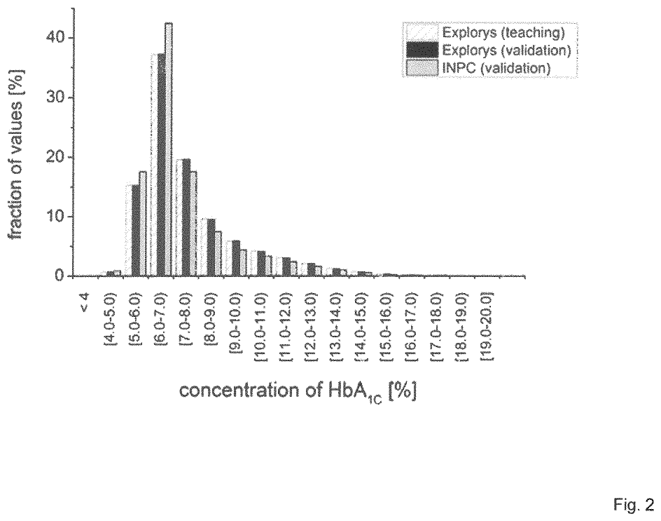

FIG. 2 the distribution of HbA1C in an example teaching training set, validation set and further validation set;

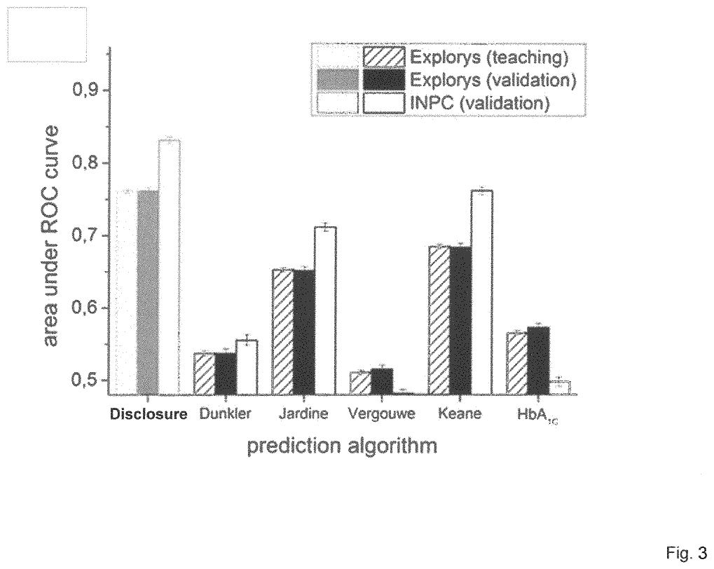

FIG. 3 a comparison of algorithms for predicting CKD;

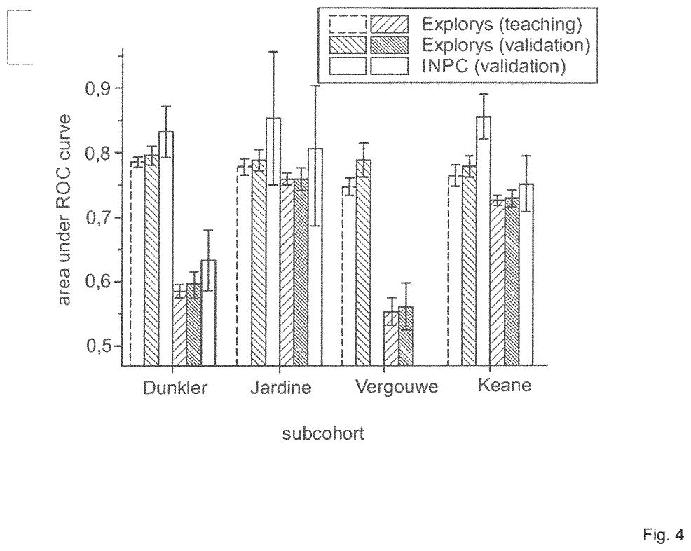

FIG. 4 a comparison of algorithms for predicting CKD using subcohorts;

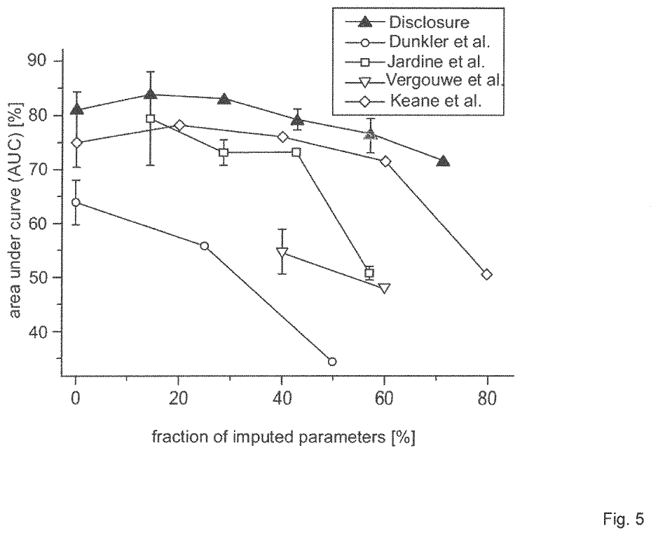

FIG. 5 another comparison of algorithms for predicting CKD; and

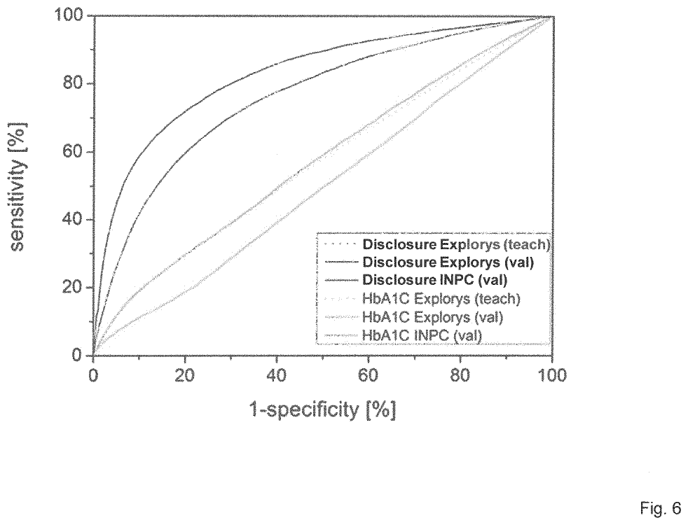

FIG. 6 a further comparison of algorithms for predicting CKD.

In general, in any of the embodiments of the method for screening a subject for the risk of CKD, creatinineₘₐₓ may be a maximum sample level of creatinine from a plurality of sample levels of creatinine for the subject, albuminₘᵢₙ may be a minimum sample level of albumin from a plurality of sample levels of albumin for the subject, eGFRₘᵢₙ may be a minimum sample level of estimated glomerular filtration rate from a plurality of sample levels of estimated glomerular filtration rate for the subject, BMIₘᵢₙ may be a minimum value of the Body Mass Index (BMI) from a plurality of values of the BMI for the subject, Glucoseₘᵢₙ may be a minimum sample level of glucose from a plurality of sample levels of glucose for the subject and HbAₘₑₐₙ may be a mean sample level of C-fraction of glycated haemoglobin A1 from a plurality of sample levels of C-fraction of glycated haemoglobin A1 for the subject. Such values and/or sample levels may be determined from values and/or sample levels already on file for the subject. Alternatively or in addition, values and/or sample levels may be determined for the subject specifically for use with the method for screening a subject for the risk of CKD. Values and/or sample levels may be real world data, i.e., unlike clinical data, they may not be restricted regarding, for example, completeness or veracity of the data.

In the method for screening a subject for the risk of CKD, creatinineₘₐₓ may be expressed in units of mg/dl, albuminₘᵢₙ may be expressed in units of g/dl, eGFRₘᵢₙ may be expressed in units of ml/min/1.73 m², BMIₘᵢₙ may be expressed in units of kg/m², Glucoseₘᵢₙ may be a expressed in units of mg/dl and HbAₘₑₐₙ may be expressed in units of %. Glomerular filtration rates may be estimated using an MDRD formula, known in the art as such. Alternatively, glomerular filtration rates may be estimated using the CKD-EPI formula, known in the art as such.

Marker data may be received for a subject suffering from diabetes. In alternative, the subject does not suffer from diabetes but may is at risk of suffering from diabetes in the future. The marker data is indicative for marker parameters age, creatinineₘₐₓ and albuminₘᵢₙ for the subject. The parameter “age” indicates the age of the subject in years. The parameter “creatinineₘₐₓ” is indicative of a maximum sample level of creatinine from a plurality of sample levels of creatinine on file for the subject and collected over the prior 2 years from blood samples. The parameter “albuminₘᵢₙ” is indicative of a minimum sample level of albumin from a plurality of sample levels of albumin on file for the subject and collected over the prior 2 years from blood samples.

According to this embodiment, marker data is indicative for the marker parameters age, creatinineₘₐₓ and albuminₘᵢₙ for the subject, thereby providing a simplified method for calculating a risk factor indicative of the risk of suffering CKD for the subject. In further embodiments, as will be set forth in more detail below, further marker data indicative for at least one of the marker parameters eGFRₘᵢₙ, BMIₘᵢₙ, Glucoseₘᵢₙ and HbAₘₑₐₙ for the subject may be included in the calculation to provide a more accurate calculation for the risk factor.

In an example, a risk factor indicative of the risk of suffering CKD for the subject is determined from the plurality of marker parameters according to the following equations:

$${P}_{CKD} = \frac{{e}^{{P}_{CKD\_Pred}}}{1 + {e}^{{P}_{CKD\_Pred}} + {e}^{{P}_{Death\_Pred}}}$$

$${P}_{CKD\_Pred} = 0.02739 \cdot age \text{/} year + 1.387 \cdot {creatinine}_{max} \cdot dl \text{/} mg - 0.3356 \cdot {albumin}_{min} \cdot dl \text{/} g - 3.1925$$

$${P}_{{Death}_{-} Pred} = 0.06103 \cdot age \text{/} year + 0.8194 \cdot {creatinine}_{max} \cdot dl \text{/} mg - 0.9336 \cdot {albumin}_{min} \cdot dl \text{/} g - 3.3325$$

Thereby, the age value is weighted higher than the sample level of albumin and the sample level of creatinine is weighted higher than the sample level of albumin.

Marker data may be received for a subject suffering from diabetes. In alternative, the subject does not suffer from diabetes but may is at risk of suffering from diabetes in the future. The marker data is indicative for marker parameters age, creatinineₘₐₓ, albuminₘᵢₙ and eGFRₘᵢₙ for the subject. The parameter “age” indicates the age of the subject in years. The parameter “creatinineₘₐₓ” is indicative of a maximum sample level of creatinine from a plurality of sample levels of creatinine on file for the subject and collected over the prior 2 years from blood samples. The parameter “albuminₘᵢₙ” is indicative of a minimum sample level of albumin from a plurality of sample levels of albumin on file for the subject and collected over the prior 2 years from blood samples. The parameter “eGFRₘᵢₙ” is indicative of a minimum sample level of estimated glomerular filtration rate from a plurality of sample levels of estimated glomerular filtration rate on file for the subject and collected over the prior 2 years.

In an example, a risk factor indicative of the risk of suffering CKD for the subject is determined from the plurality of marker parameters according to the following equations:

$${P}_{CKD} = \frac{{e}^{{P}_{CKD\_Pred}}}{1 + {e}^{{P}_{CKD\_Pred}} + {e}^{{P}_{Death\_Pred}}}$$

$${P}_{CKD\_Pred} = 0.02739 \cdot age \text{/} year + 1.387 \cdot {creatinine}_{max} \cdot dl \text{/} mg - 0.3356 \cdot {albumin}_{min} \cdot dl \text{/} g - 0.02843 \cdot {eGFR}_{min} \cdot min \cdot 1.73 {m}^{2} \text{/} ml - 1.3013$$

$${P}_{{Death}_{-} Pred} = 0.06103 \cdot age \text{/} year + 0.8194 \cdot {creatinine}_{max} \cdot dl \text{/} mg - 0.9336 \cdot {albumin}_{min} \cdot dl \text{/} g + 0.01654 \cdot {eGFR}_{min} \cdot min \cdot 1.73 {m}^{2} \text{/} ml - 4.4328$$

Thereby, the age value is weighted higher than the sample level of albumin, the sample level of creatinine is weighted higher than the sample level of albumin and each of the age value, the sample level of albumin, and the sample level of creatinine are weighted higher than the sample level of glomerular filtration rate.

Marker data may be received for a subject suffering from diabetes. In alternative, the subject does not suffer from diabetes but may is at risk of suffering from diabetes in the future. The marker data is indicative for marker parameters age, creatinineₘₐₓ, albuminₘᵢₙ, eGFRₘᵢₙ, BMIₘᵢₙ, Glucoseₘᵢₙ and HbAₘₑₐₙ for the subject. The parameter “age” indicates the age of the subject in years. The parameter “creatinineₘₐₓ” is indicative of a maximum sample level of creatinine from a plurality of sample levels of creatinine on file for the subject and collected over the prior 2 years from blood samples. The parameter “albuminₘᵢₙ” is indicative of a minimum sample level of albumin from a plurality of sample levels of albumin on file for the subject and collected over the prior 2 years from blood samples. The parameter “eGFRₘᵢₙ” is indicative of a minimum sample level of estimated glomerular filtration rate from a plurality of sample levels of estimated glomerular filtration rate on file for the subject and collected over the prior 2 years. The parameter “BMIₘᵢₙ” is indicative of a minimum value for the Body Mass Index from a plurality of values for the Body Mass Index on file for the subject and collected over the prior 2 years. The parameter “Glucoseₘᵢₙ” is indicative of a minimum sample level of blood glucose from a plurality of sample levels of blood glucose on file for the subject and collected over the prior 2 years. The parameter “HbAₘₑₐₙ” is indicative of a mean sample level of C-fraction of glycated haemoglobin A1 from a plurality of sample levels of C-fraction of glycated haemoglobin A1 on file for the subject and collected over the prior 2 years.

A risk factor indicative of the risk of suffering CKD for the subject is determined from the plurality of marker parameters according to the following equations:

$${P}_{CKD} = \frac{{e}^{{P}_{CKD\_Pred}}}{1 + {e}^{{P}_{CKD\_Pred}} + {e}^{{P}_{Death\_Pred}}}$$

$${P}_{CKD\_Pred} = 0.02739 \cdot age \text{/} year + 1.387 \cdot {creatinine}_{max} \cdot dl \text{/} mg - 0.3356 \cdot {albumin}_{min} \cdot dl \text{/} g - 0.02843 \cdot {eGFR}_{min} \cdot min \cdot 1.73 {m}^{2} \text{/} ml + 0.01128 \cdot {BMI}_{min} + 0.0004946 \cdot {Glucose}_{min} \cdot dl \text{/} mg + 0.0893 \cdot {HbA}_{mean} / \% - 2.409$$

$${P}_{{Death}_{-} Pred} = 0.06103 \cdot age \text{/} year + 0.8194 \cdot {creatinine}_{max} \cdot dl \text{/} mg - 0.9336 \cdot {albumin}_{min} \cdot dl \text{/} g + 0.01654 \cdot {eGFR}_{min} \cdot min \cdot 1.73 {m}^{2} \text{/} ml - 0.0101 \cdot {BMI}_{min} + 0.0009107 \cdot {Glucose}_{min} \cdot dl \text{/} mg + 0.04368 \cdot {HbA}_{mean} / \% - 4.557$$

Thereby, the age value is weighted higher than the sample level of albumin, the age is weighted higher than the sample level of creatinine, the sample level of creatinine is weighted higher than the sample level of albumin and each of the age value, the sample level of albumin, and the sample level of creatinine are weighted higher than the sample level of glomerular filtration rate. Further, each of the age value, the sample level of albumin, the sample level of creatinine and the sample level of glomerular filtration rate are weighted higher than each of the value of the Body Mass Index, the sample level of of blood glucose and the sample level of C-fraction of glycated haemoglobin A1.

In the method for screening a subject for the risk of CKD, all or any of the values to be multiplied with the values and/or sample levels for the subject in determining P_{CKD_Pred} and/or P_{Death_Pred} may be determined as follows.

An algorithm is taught using electronic health record (EHR) data, for example from 417,912 people with diabetes (types 1 and 2) among more than 55 million people represented in a database. The data is retrieved for the time window starting 2 years before the initial diagnosis of diabetes and lasting until up to 3 years following this diagnosis. The data can be considered as real-world data (RWD) and no general restrictions on, for example, completeness or veracity of the data are applied. Missing data is imputed with the cohort's mean value before feature selection and teaching the algorithm. Logistic regression is chosen for teaching rather than a black box approach such as deep learning. This may allow for the medical interpretation of the data-driven analysis. After teaching, an independent sample set of data, for example originating from 104,504 further individuals in the same database, is used for independent validation. In addition, the algorithm is applied to data, for example from 82,912 persons with type-2 diabetes included in a further database.

ICD codes may be used as target variables for training as well as the CKD reference diagnosis in the analysis of the validation results. The definition of the target feature “CKD” may be solely based on the occurrence of the respective ICD codes in the databases. In order to maintain the RWD character of the data set, no additions or changes may be made to the databases. Such ICD codes may comprise ICD-9 codes and ICD-10 codes, for example the following ICD codes: 250.40, 250.41, 250.42, 250.43, 585.1, 585.2, 585.3, 585.4, 585.5, 585.6, 585.9, 403.00, 403.01, 403.11, 403.90, 403.91, 404.0, 404.00, 404.01, 404.02, 404.03, 404.1, 404.10, 404.11, 404.12, 404.13, 404.9, 404.90, 404.91, 404.92, 404.93, 581.81, 581.9, 583.89, 588.9, E10.2, E10.21, E10.22, E10.29, E11.2, E11.21, E11.22, E11.29, N17.0, N17.1, N17.2, N17.8, N17.9, N18.1, N18.2, N18.3, N18.4, N18.5, N18.6, N18.9, N19, I12.0, I12.9, I13, I13.0, I13.1, I13.10, I13.11, I13.2, N04.9, N05.8, N08 and/or N25.9.

The ICD-9 codes 250.40, 403.90, 585.3, 585.9 may be the most abundant diagnosis in the respective time windows of the data set and they occur in >5% of the cases within each of the data sets.

In a further method for screening a subject for the risk of CKD, all or any of the values to be multiplied with the values and/or sample levels for the subject in determining P_{CKD_Pred} and/or P_{Death_Pred} may be determined as follows.

In order to allow an early risk assessment for CKD, EHR data is extracted from a database, which includes longitudinal data originating from more than 55 million patients with thousands of person-specific features. The data extracted from the database for the investigation originates from 522,416 people newly diagnosed with diabetes. The data is retrieved for the time window starting 2 years before the initial diagnosis of diabetes and lasting until up to 3 years following this diagnosis. People with prior renal dysfunctions are excluded in order to perform an unbiased risk assessment for the later development of CKD. Following the guidelines for the diagnosis of diabetes, it is requested that the concentration of the β-N-1-deoxyfructosyl component of hemoglobin (HbA1C), an important clinical laboratory parameter in diabetes diagnosis and treatment, was determined at least once prior to (or within 7 days after) the initial diagnosis of diabetes. The data selected from the database can be considered as RWD because no further restrictions on the completeness or veracity of the data are applied. In order to cope with these challenges arising from the use of RWD the following approach may be implemented:
1. The data selected from the database is randomly split into a teaching set (417,912 people) and a validation set (104,504 people).
2. Features are selected on the basis of a data-driven correlation analysis within the teaching set and cross-checked for conceptual (especially medical) relevance.
3. Missing values are imputed with the dataset's mean value. Optionally, a screening or determination of outlier values has been performed prior to teaching. In case of determining an outlier, the value has been substituted by an appropriate value (If the feature value is higher than the upper limit of the specific allowable range for that feature, the value can be replaced by the upper limit of that range before using it in the prediction formula. If the feature value is lower than the lower limit of the specific allowable range for that feature, the value can be replaced by the lower limit before using it in the prediction formula).
4. The risk predictor is taught exclusively in this RWD's teaching set.
5. After the teaching is completed, the validation set is subjected to the algorithm in order to assess the quality of the algorithm. No further readjustment of the algorithm is performed.
6. In addition, RWD from 82,912 people represented in a further database is used as a further, independent validation set.

Analysis of an example teaching training set (from the IBM Explorys database; see Kaelber, D. C. et al., Patient characteristics associated with venous thromboembolic events: a cohort study using pooled electronic health record data, J Am Med Inform Assoc 19, 965-972, 2012), validation set (from the IBM Explorys database) and further validation set (from the Indiana Network for Patient Care (INPC); see McDonald, C. J. et al., The Indiana Network for Patient Care: a working local health information infrastructure, Health Affairs 24, 1214-1220, 2005) has been conducted. In the teaching logistic regression has been applied.

In the teaching and validation sets, 50.7%, 50.9% and 51.7% of the persons, respectively, are female. The median age of each population is 60 years, 60 years, and 59 years, respectively. The median concentrations of HbA1C are 6.8%, 6.8%, and 6.6%, respectively. The distributions of age and HbA1C are shown in FIGS. 1 and 2, respectively.

In certain embodiments, for feature selection, almost 300 features are initially chosen based on medical as well as data-driven criteria. This feature set is then culled in multiple steps. Observational features that are defined for less than half of the patients in the cohort are removed, as are outliers of continuous features. Categorical features with 99% of occurrences in a single category and continuous features with a standard deviation of 0.001% are not considered. Finally, only those features which already showed correlation with the diagnosis of CKD in a univariate analysis as quantified by Pearson's chi-squared coefficient χ²>0.95 are retained. For predictive analysis, a logistic regression model based on forward selection (see Bursac, Z. et al., Purposeful selection of variables in logistic regression, Source code for biology and medicine 3, 17, 2008; and Hosmer Jr., D. W. et al., Applied logistic regression, Vol. 398, John Wiley & Sons, 2013) is trained on the teaching set and delivers the person's age, body mass index, glomerular filtration rate and the concentrations of glucose, albumin, and creatinine as the most prominent parameters. An assessment of the medical relevance of these features may be performed to ensure clinical applicability, in contrast to a “black box” approach based on, for example, deep learning. HbA_{1C} may be added to the top-7 feature list in order to reflect current state-of-the-art methods. The teaching of algorithms may be based on correlation, but may not infer any causality. After teaching, the algorithm is applied to the two independent datasets, namely the validation sets.

ICD codes may be used as target variables for training as well as the CKD reference diagnosis in the analysis of the validation results. The definition of the target feature “CKD” may be solely based on the occurrence of the respective ICD codes in the databases. In order to maintain the RWD character of the data set, no additions or changes may be made to the databases. Such ICD codes may comprise ICD-9 codes and ICD-10 codes, for example the following ICD codes: 250.40, 250.41, 250.42, 250.43, 585.1, 585.2, 585.3, 585.4, 585.5, 585.6, 585.9, 403.00, 403.01, 403.11, 403.90, 403.91, 404.0, 404.00, 404.01, 404.02, 404.03, 404.1, 404.10, 404.11, 404.12, 404.13, 404.9, 404.90, 404.91, 404.92, 404.93, 581.81, 581.9, 583.89, 588.9, E10.2, E10.21, E10.22, E10.29, E11.2, E11.21, E11.22, E11.29, N17.0, N17.1, N17.2, N17.8, N17.9, N18.1, N18.2, N18.3, N18.4, N18.5, N18.6, N18.9, N19, I12.0, I12.9, I13, I13.0, I13.1, I13.10, I13.11, I13.2, N04.9, N05.8, N08 and/or N25.9.

In an embodiment, the ICD-9 codes 250.40, 403.90, 585.3, 585.9 are the most abundant diagnosis in the respective time windows of the data set and they occur in >5% of the cases within each of the data sets.

Following, experimental data are discussed.

The area under the receiver operating characteristic (compare Swets, J. A., Measuring the accuracy of diagnostic systems, Science 240, 1285-1293, 1988) curve (AUC) is frequently used to measure the quality of clinical markers as well as machine learning algorithms (see Bradley, A. P., The use of the area under the ROC curve in the evaluation of machine learning algorithms, Pattern Recognition 30, 1145-1159, 1997). A perfect marker would achieve AUC=1.0, whereas flipping a coin would result in AUC=0.5. After teaching the model (based on Explorys) according to the present disclosure using the seven most promising features, the AUC of the prediction algorithm amounted to 0.7937 (0.790 . . . 0.797) when applied to the overall independent validation data (Explorys: 0.761, INPC: 0.831).

The AUC increased to 0.7939 and 0.7967 if the top-10 and top-12 features were used for evaluation, respectively. In turn, a simple HbA1C model (see The Diabetes Control and Complications Trial Research Group. The effect of intensive treatment of diabetes on the development and progression of long-term complications in insulin-dependent diabetes mellitus, N Engl J Med 329, 977-986, 1993) yielded 0.483 (0.477 . . . 0.489) for the same datasets. The algorithm according to the present disclosure therefore outperforms risk predictors using HbA1C alone for people newly diagnosed with diabetes.

In further analysis, the algorithm according to the present disclosure was compared to published algorithms derived from data sourced from major clinical studies such as the ONTARGET, ORIGIN, RENAAL and ADVANCE studies (cf. Dunkler, D. et al., Risk Prediction for Early CKD in Type 2 Diabetes, Clin J Am Soc Nephrol 10, 1371-1379, 2015; Vergouwe, Y. et al., Progression to microalbuminuria in type 1 diabetes: development and validation of a prediction rule, Diabetologia 53, 254-262, 2010; Keane, W. F. et al., Risk Scores for Predicting Outcomes in Patients with Type 2 Diabetes and Nephropathy: The RENAAL Study, Clin J Am Soc Nephrol 1, 761-767, 2006; and Jardine, M. J. et al., Prediction of Kidney-Related Outcomes in Patients With Type 2 Diabetes, Am J Kidney Dis. 60, 770-778, 2012). As shown in FIG. 3, the algorithm according to the present disclosure outperformed each of these algorithms for all RWD cohorts. While this finding is important in terms of applicability and relevance in everyday settings, it may be argued that the validity of the published algorithms is limited to the inclusion and exclusion criteria of the corresponding clinical studies. Therefore, subcohorts of the IBM Explorys and INPC databases were formed according to the selection criteria of these studies, and the algorithm according to the present disclosure (without any retraining) was benchmarked against the literature algorithms solely for these subcohorts. Although the AUCs of the published algorithms increased for all specific subcohorts as expected, the superiority of the RWD-trained model according to the present disclosure prevailed (FIG. 4). However, the inclusion and exclusion criteria for the subcohorts could not be met precisely in all cases for the present RWD set because the clinical studies demanded some information which is not available in the database (e.g. waist-to-hip ratio). In addition, there were differences in the choice of the complication incidence time window. Nevertheless, the features that were prioritized for classification with the algorithm according to the present disclosure are similar to those reported in the literature, thus further bolstering the algorithm's validity.

The use of RWD and in particular the inclusion of incomplete or erroneous data in the training set for the algorithm according to the present disclosure constitutes a major difference compared to clinical study-based algorithms. The imputation of missing data provides a typical example of predictive analytics in RWD cohorts, whereas imputation would be inconceivable in a clinical study setting. To further elucidate the role of imputation, the algorithm according to the present disclosure was applied to RWD solely representing individuals providing a complete set of information (i.e. no imputation was necessary). In this case, the AUCs remained comparable to the previous values for the overall RWD set, that is 0.792 (0.787 . . . 0.797), 0.791 (0.780 . . . 0.801), and 0.809 (0.769 . . . 0.846) for the Explorys teaching training set, the Explorys validation set, and the INPC validation set, respectively. Further analysis revealed the rapid loss of classification accuracy with an increasing fraction of imputed data when the earlier algorithms were tested, whereas the algorithm according to the present disclosure achieved much higher stability, even for higher proportions of imputed data (FIG. 5). It is concluded that—at least in the present example—the teaching training of predictive analytics algorithms using RWD could achieve equivalent or even enhanced accuracy compared to clinical trial data, but further testing on additional datasets will be necessary before these conclusions can be generalised.

In summary, it is demonstrated that a predictive algorithm for CKD performed significantly better in individuals newly diagnosed with diabetes if trained on RWD rather than clinical study data. This statement held true when the algorithm according to the present disclosure was applied to the overall RWD cohort as well as specific subcohorts as defined by the corresponding clinical studies. The results support the path towards high-quality predictive models that can be applied in a clinical setting, enabling the shift towards personalized and outcome-based healthcare.

The performance of a method for screening a subject for the risk of CKD or for identifying those people at high risk of developing CKD may be judged according to sensitivity (fraction of correctly predicted high-risk patients) and specificity (fraction of correctly assigned low-risk patients). However, either of these numbers can be improved at the expense of the other simply by changing the threshold between high and low risk. Hence, data pairs of sensitivity and specificity may be illustrated in forms of the so-called receiver operating characteristic (ROC) curve (see Swets, J. A., Measuring the accuracy of diagnostic systems, Science 240, 1285-1293, 1988) in which the sensitivity is plotted as a function of 1-specificity (which corresponds to the fraction of falsely assigned high-risk persons). The ROC curve of the risk model according to the present disclosure is shown for the Explorys training set, the Explorys validation set and the INPC validation set in FIG. 6 together with the corresponding ROC curves for a model based solely on HbA1C.

For a perfect classifier, the ROC curve reaches the upper-left corner. In fact, the threshold corresponding to the data pair closest to this corner is dubbed the “optimal threshold”. When aiming for high sensitivity, an alternative threshold may be chosen to guarantee a sensitivity of, for example, 90%. The corresponding results are summarized in the following Table together with the positive predictive value (PPV) and negative predictive value (NPV). Similar measures from the field of bioinformatics—namely accuracy and F-score (Van Rijsbergen, C. J., Information Retrieval, Butterworth-Heinemann Newton, Mass., USA, 1979)—supplement the list of examples in the Table 2.

TABLE 2

| | | Cohort | sensitivity | specificity | PPV | NPV | acc. | F-measure |
|---|---|---|---|---|---|---|---|---|
| a) | HbA1c | Explorys | 53.5 | 55.1 | 11.7 | 91.4 | 55.0 | 19.2 |
| | | (teach) | | | | | | |
| | | Explorys (val) | 54.4 | 55.2 | 11.9 | 91.6 | 55.1 | 19.5 |
| | | INPC (val) | 37.5 | 61.5 | 11.3 | 88.2 | 58.7 | 17.4 |
| b) | present | Explorys | 68.2 | 72.6 | 21.7 | 95.4 | 72.1 | 32.9 |
| | model | (teach) | | | | | | |
| | | Explorys (val) | 68.3 | 72.4 | 21.6 | 95.3 | 72.0 | 32.8 |
| | | INPC (val) | 79.3 | 71.2 | 26.6 | 96.3 | 72.2 | 39.8 |
| c) | present | Explorys | (90.0) | 35.0 | 13.3 | 96.9 | 40.5 | 23.2 |
| | model* | (teach) | | | | | | |
| | | Explorys (val) | 90.0 | 34.9 | 13.3 | 96.9 | 40.4 | 23.2 |
| | | INPC (val) | 95.3 | 27.6 | 14.7 | 97.8 | 35.5 | 25.5 |

A comparative evaluation of the full algorithm according to the present disclosure (seven values/sample levels for the subject, missing values/sample levels imputed) to a reduced algorithm according to the present disclosure (age, creatinineₘₐₓ and albuminₘᵢₙ for the subject, population mean values for the remaining values/sample levels), respectively applied to INPC data, has resulted in an AUC of 0.831 (confidence interval 0.827 to 0.836) for the full algorithm and an AUC of 0.823 (confidence interval 0.818 to 0.827) for the reduced algorithm. Therefore, even with the reduced algorithm, useful predictions may be achieved.

1. A method for screening a subject for the risk of chronic kidney disease (CKD), comprising
receiving marker data indicative for a plurality of marker parameters for a subject, such plurality of marker parameters indicating, for the subject for a measurement period, an age value, a sample level of creatinine, and a sample level of albumin; and
determining a risk factor indicative of the risk of suffering CKD for the subject from the plurality of marker parameters, wherein the determining comprises
weighting the age value higher than the sample level of albumin, and
weighting the sample level of creatinine higher than the sample level of albumin.

2. The method according to claim 1, further comprising the plurality of marker parameters indicating, for the subject, a blood sample level of creatinine.

3. The method according to claim 1, further comprising the plurality of marker parameters indicating, for the subject, a blood sample level of albumin.

4. The method according to claim 1, wherein the subject is a diabetes patient.

5. The method according to claim 1, wherein the measurement period is limited to two years.

6. The method according to claim 1, wherein the subject has not been diagnosed with diabetes by the end of the measurement period.

7. The method according to claim 4, wherein the measurement period lies after a diabetes diagnosis for the subject, at least in part.

8. The method according to claim 1, wherein the risk factor is indicative of the risk of suffering CKD for the subject within a prediction time period of three years from the end of the measurement period.

9. The method according to claim 1, wherein the determining further comprises weighting the age higher than the sample level of creatinine.

10. The method according to claim 1, wherein the receiving comprises receiving marker data indicative for a plurality of marker parameters for a subject having a sample level of HbA1c of less than 6.5%.

11. The method according to claim 1, further comprising
the plurality of marker parameters indicating, for the subject, a sample level of a glomerular filtration rate; and
in the determining, weighting each of the age value, the sample level of albumin, and the sample level of creatinine higher than the sample level of a glomerular filtration rate.

12. The method according to claim 1, wherein the risk factor is determined according to the equation
$${P}_{CKD} = \frac{{e}^{{P}_{CKD\_Pred}}}{1 + {e}^{{P}_{CKD\_Pred}}} ,$$
and wherein
P_{CKD} is the risk factor;
P_{CKD_Pred}=c_{CKD1}·age+c_{CKD2}·creatinine+c_{CKD}3·albumin+c_{CKD4};
age is the age of the subject;
creatinine is a sample level of creatinine for the subject;
albumin is a sample level of albumin for the subject; and
c_{CKD1}, c_{CKD2}, c_{CKD3}, and c_{CKD4} are constants.

13. The method according to claim 1, wherein the risk factor is determined according to the equation
$${P}_{CKD}^{′} = \frac{{e}^{{P}_{CKD\_Pred}^{′}}}{1 + {e}^{{P}_{CKD\_Pred}^{′}}} ,$$
and wherein
P′_{CKD} is the risk factor;
P′_{CKD_Pred}=c′_{CKD1}·age+c′_{CKD2}·creatinine+c′_{CKD3}·albumin+c′_{CKD4}+c′_{CKD5}·eGFR;
age is the age of the subject;
creatinine is a sample level of creatinine for the subject;
albumin is a sample level of albumin for the subject;
eGFR is a sample level of estimated glomerular filtration rate for the subject; and
c′_{CKD1}, c′_{CKD2}, c′_{CKD3}, c′_{CKD4}, and c′_{CKD5} are constants.

14. A computer-implemented method for screening a subject for the risk of chronic kidney disease (CKD) in a data processing system having a processor and a non-transitory memory storing a program causing the processor to execute:
receiving marker data indicative for a plurality of marker parameters for a subject, such plurality of marker parameters indicating, for the subject for a measurement period, an age value, a sample level of albumin, and a sample level of creatinine; and
determining a risk factor indicative of the risk suffering CKD for the subject from the plurality of marker parameters, wherein the determining comprises
weighting the age value higher than the sample level of albumin, and
weighting the sample level of creatinine higher than the sample level of albumin.

15. A method for screening a subject for the risk of chronic kidney disease (CKD), comprising
receiving marker data indicative for a plurality of marker parameters, such plurality of marker parameters indicating
an age value for the subject,
a sample level of creatinine for a measurement period, and
a sample level of albumin for a measurement period; and
determining a risk factor indicative of the risk of suffering CKD for the subject from the plurality of marker parameters, wherein the determining comprises
weighting the age value higher than the sample level of albumin, and
weighting the sample level of creatinine higher than the sample level of albumin, wherein at least one of the sample level of creatinine and the sample level of albumin is indicative of a generalized value of sample levels for a reference group of subjects not comprising the subject, for a respective measurement period of each subject of the reference group of subjects.
